SEQUENCE LISTING (21 sequences)

SEQ ID NO: 1 - protein, 447 aa, Artificial
  genotype 1b Y93H NS5A
SGSWLRDVWDWICTVLTDFKTWLQSKLLPRLPGVPFLSCQRGYKGVWRGDGIMQTTCPCGAQIAGHVKNGSMRIVGPRTCSNTWHGTFPINAHTTGPCTPSPAPNYSRALWRVAAEEYVEVTRVGDFHYVTGMTTDNVKCPCQVPAPEFFTEVDGVRLHRYAPACKPLLREDVTFQVGLNQYLVGSQLPCEPEPDVTVLTSMLTDPSHITAETAKRRLARGSPPSLASSSAIQLSAPSLKATCTTHHDSPDADLIEANLLWRQEMGGNITRVESENKVVILDSFEPLHAEGDEREISVAAEILRKSRKFPSALPIWARPDYNPPLLESWKDPDYVPPVVHGCPLPPTKAPPIPPPRRKRTVVLTESNVSSALAELATKTFGSSGSSAVDSGTATALPDLASDDGDKGSDVESYSSMPPLEGEPGDPDLSDGSWSTVSEEASEDVVCC

SEQ ID NO: 2 - protein, 447 aa, Artificial
  genotype 1b L31V NS5A
SGSWLRDVWDWICTVLTDFKTWLQSKLLPRVPGVPFLSCQRGYKGVWRGDGIMQTTCPCGAQIAGHVKNGSMRIVGPRTCSNTWHGTFPINAYTTGPCTPSPAPNYSRALWRVAAEEYVEVTRVGDFHYVTGMTTDNVKCPCQVPAPEFFTEVDGVRLHRYAPACKPLLREDVTFQVGLNQYLVGSQLPCEPEPDVTVLTSMLTDPSHITAETAKRRLARGSPPSLASSSAIQLSAPSLKATCTTHHDSPDADLIEANLLWRQEMGGNITRVESENKVVILDSFEPLHAEGDEREISVAAEILRKSRKFPSALPIWARPDYNPPLLESWKDPDYVPPVVHGCPLPPTKAPPIPPPRRKRTVVLTESNVSSALAELATKTFGSSGSSAVDSGTATALPDLASDDGDKGSDVESYSSMPPLEGEPGDPDLSDGSWSTVSEEASEDVVCC

SEQ ID NO: 3 - protein, 447 aa, Artificial
  genotype 1b L31V & Y93H NS5A
SGSWLRDVWDWICTVLTDFKTWLQSKLLPRVPGVPFLSCQRGYKGVWRGDGIMQTTCPCGAQIAGHVKNGSMRIVGPRTCSNTWHGTFPINAHTTGPCTPSPAPNYSRALWRVAAEEYVEVTRVGDFHYVTGMTTDNVKCPCQVPAPEFFTEVDGVRLHRYAPACKPLLREDVTFQVGLNQYLVGSQLPCEPEPDVTVLTSMLTDPSHITAETAKRRLARGSPPSLASSSAIQLSAPSLKATCTTHHDSPDADLIEANLLWRQEMGGNITRVESENKVVILDSFEPLHAEGDEREISVAAEILRKSRKFPSALPIWARPDYNPPLLESWKDPDYVPPVVHGCPLPPTKAPPIPPPRRKRTVVLTESNVSSALAELATKTFGSSGSSAVDSGTATALPDLASDDGDKGSDVESYSSMPPLEGEPGDPDLSDGSWSTVSEEASEDVVCC

SEQ ID NO: 4 - protein, 448 aa, Artificial
  genotype 1a M28T NS5A
SGSWLRDIWDWICEVLSDFKTWLKAKLTPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 5 - protein, 448 aa, Artificial
  genotype 1a Q30R NS5A
SGSWLRDIWDWICEVLSDFKTWLKAKLMPRLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 6 - protein, 448 aa, Artificial
  genotype 1a L31V NS5A
SGSWLRDIWDWICEVLSDFKTWLKAKLMPQVPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 7 - protein, 448 aa, Artificial
  genotype 1a Y93H NS5A
SGSWLRDIWDWICEVLSDFKTWLKAKLMPQLPGIPFVSCQRGYRGVWRGDGIMHTRCHCGAEITGHVKNGTMRIVGPRTCRNMWSGTFPINAHTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 8 - protein, 448 aa, Artificial
  genotype 1a Q30H NS5A
SGSWLRDIWDWICEVLSDFKTWLKAKLMPHLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 9 - protein, 448 aa, Artificial
  genotype 1a Y93N NS5A
SGSWLRDIWDWICEVLSDFKTWLKAKLMPQLPGIPFVSCQRGYRGVWRGDGIMHTRCHCGAEITGHVKNGTMRIVGPRTCRNMWSGTFPINANTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 10 - protein, 448 aa, Artificial
  genotype 1a Q30E NS5A
SGSWLRDIWDWICEVLSDFKTWLKAKLMPELPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 11 - protein, 448 aa, Artificial
  genotype 1a L31V & Y93H NS5A
SGSWLRDIWDWICEVLSDFKTWLKAKLMPQVPGIPFVSCQRGYRGVWRGDGIMHTRCHCGAEITGHVKNGTMRIVGPRTCRNMWSGTFPINAHTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 12 - protein, 448 aa, Artificial
  genotype 1a M28T & Q30H NS5A
SGSWLRDIWDWICEVLSDFKTWLKAKLTPHLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 13 - protein, 448 aa, Artificial
  genotype 1a Q30R & H58D NS5A
SGSWLRDIWDWICEVLSDFKTWLKAKLMPRLPGIPFVSCQRGYRGVWRGDGIMHTRCDCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 14 - protein, 448 aa, Artificial
  genotype 1a Q30H & Y93H NS5A
SGSWLRDIWDWICEVLSDFKTWLKAKLMPHLPGIPFVSCQRGYRGVWRGDGIMHTRCHCGAEITGHVKNGTMRIVGPRTCRNMWSGTFPINAHTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 15 - protein, 448 aa, Artificial
  genotype 1a Q30R & E62D NS5A
SGSWLRDIWDWICEVLSDFKTWLKAKLMPRLPGIPFVSCQRGYRGVWRGDGIMHTRCHCGADITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 16 - protein, 448 aa, Artificial
  genotype 1a wild type NS5A
SGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSAIQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCC

SEQ ID NO: 17 - protein, 466 aa, Artificial
  genotype 2a wild type NS5A sequence  =  genotype 2a (JFH) NS5A
SGSWLRDVWDWVCTILTDFKNWLTSKLFPKLPGLPFISCQKGYKGVWAGTGIMTTRCPCGANISGNVRLGSMRITGPKTCMNTWQGTFPINCYTEGQCAPKPPTNYKTAIWRVAASEYAEVTQHGSYSYVTGLTTDNLKIPCQLPSPEFFSWVDGVQIHRFAPTPKPFFRDEVSFCVGLNSYAVGSQLPCEPEPDADVLRSMLTDPPHITAETAARRLARGSPPSEASSSVSQLSAPSLRATCTTHSNTYDVDMVDANLLMEGGVAQTEPESRVPVLDFLEPMAEEESDLEPSIPSECMLPRSGFPRALPAWARPDYNPPLVESWRRPDYQPPTVAGCALPPPKKAPTPPPRRRRTVGLSESTISEALQQLAIKTFGQPPSSGDAGSSTGAGAAESGGPTSPGEPAPSETGSASSMPPLEGEPGDPDLESDQVELQPPPQGGGVAPGSGSGSWSTCSEEDDTTVCC

SEQ ID NO: 18 - protein, 447 aa, Artificial
  genotype 3a wild type NS5A
SGDWLRIIWDWVCSVLSDFKTWLSAKIMPALPGLPFISCQKGYKGVWRGDGVMSTRCPCGASISGHVKNGSMRLAGPRSCANMWHGTFPINEYTTGPSTPSPAPNYSRALWRVAAEEYVEVTRVGDFHYVTGMTTDNVKCPCQVPAPEFFTEVDGVRLHRYAPACKPLLREEVTFLVGLNQYLVGSQLPCEPEPDVAVLTSMLTDPSHITAETAKRRLARGSPPSLASSSAIQLSAPSLKATCTTRHDSPDADLIEANLLWRQEMGGNITRVESENKVVILDSFEPLQAEEDEREVSVPAEILRRSRKFPRAMPIWARPDYNPPLLESWKDPDYVPPVVHGCPLPPAKAPPIPPPRRKRTVVLSESTVSSALAELATKTFGSSESSAVDSGTATASPDQPSDDGDAGSDVESYSSMPPLEGEPGDPDLSDGSWSTVSEEASEDVVCC

SEQ ID NO: 19 - protein, 466 aa, Artificial
  genotype 2a (HC-J6CF) NS5A
GGSWLRDVWDWVCTILTDFKNWLTSKLFPKMPGLPFVSCQKGYKGVWAGTGIMTTRCPCGANISGNVRLGSMRITGPKTCMNIWQGTFPINCYTEGQCVPKPAPNFKVAIWRVAASEYAEVTQHGSYHYITGLTTDNLKVPCQLPSPEFFSWVDGVQIHRFAPTPKPFFRDEVSFCVGLNSFVVGSQLPCDPEPDTDVLMSMLTDPSHITAETAARRLARGSPPSEASSSASQLSAPSLRATCTTHGKAYDVDMVDANLFMGGDVTRIESGSKVVVLDSLDPMVEERSDLEPSIPSEYMLPKKRFPPALPAWARPDYNPPLVESWKRPDYQPATVAGCALPPPRKTPTPPPRRRRTVGLSEDSIGDALQQLAIKSFGQPPPSGDSGLSTGAGAADSGSQTPPDELALSETGSISSMPPLEGELGDPDLEPEQVEPQPPPQGGVAAPGSDSGSWSTCSEEDDSVVCC

SEQ ID NO: 20 - protein, 447 aa, Artificial
  genotype 3a Y93H NS5A
SGDWLRIIWDWVCSVLSDFKTWLSAKIMPALPGLPFISCQKGYKGVWRGDGVMSTRCPCGASISGHVKNGSMRLAGPRSCANMWHGTFPINEHTTGPSTPSPAPNYSRALWRVAAEEYVEVTRVGDFHYVTGMTTDNVKCPCQVPAPEFFTEVDGVRLHRYAPACKPLLREEVTFLVGLNQYLVGSQLPCEPEPDVAVLTSMLTDPSHITAETAKRRLARGSPPSLASSSAIQLSAPSLKATCTTRHDSPDADLIEANLLWRQEMGGNITRVESENKVVILDSFEPLQAEEDEREVSVPAEILRRSRKFPRAMPIWARPDYNPPLLESWKDPDYVPPVVHGCPLPPAKAPPIPPPRRKRTVVLSESTVSSALAELATKTFGSSESSAVDSGTVTSSPDQPSDDGDAGSDVESYSSMPPLEGEPGDPDLSDGSWSTVSEEASEDVVCC

SEQ ID NO: 21 - protein, 447 aa, Artificial
  genotype 1b wild type NS5A
SGSWLRDVWDWICTVLTDFKTWLQSKLLPRLPGVPFLSCQRGYKGVWRGDGIMQTTCPCGAQIAGHVKNGSMRIVGPRTCSNTWHGTFPINAYTTGPCTPSPAPNYSRALWRVAAEEYVEVTRVGDFHYVTGMTTDNVKCPCQVPAPEFFTEVDGVRLHRYAPACKPLLREDVTFQVGLNQYLVGSQLPCEPEPDVTVLTSMLTDPSHITAETAKRRLARGSPPSLASSSAIQLSAPSLKATCTTHHDSPDADLIEANLLWRQEMGGNITRVESENKVVILDSFEPLHAEGDEREISVAAEILRKSRKFPSALPIWARPDYNPPLLESWKDPDYVPPVVHGCPLPPTKAPPIPPPRRKRTVVLTESNVSSALAELATKTFGSSGSSAVDSGTATALPDLASDDGDKGSDVESYSSMPPLEGEPGDPDLSDGSWSTVSEEASEDVVCC

(19) United States
(12) Patent Application Publication
(10) Pub. No.: US 2013/0157894 A1
(43) Pub. Date: Jun. 20, 2013

(54) METHODS TO IDENTIFY COMBINATIONS OF NS5A TARGETING COMPOUND THAT ACT SYNERGISTICALLY TO INHIBIT HEPATITIS C VIRUS REPLICATION

(71) Applicants: Jin-Hua Sun, North Haven, CT (US); Min Gao, Madison, CT (US); Donald R. O'Boyle, II, Killingworth, CT (US); Julie A. Lemm, Durham, CT (US); Susan B. Roberts, Madison, CT (US); Makonen Belema, North Haven, CT (US); Nicholas A. Meanwell, East Hampton, CT (US)

(72) Inventors: Jin-Hua Sun, North Haven, CT (US); Min Gao, Madison, CT (US); Donald R. O'Boyle, II, Killingworth, CT (US); Julie A. Lemm, Durham, CT (US); Susan B. Roberts, Madison, CT (US); Makonen Belema, North Haven, CT (US); Nicholas A. Meanwell, East Hampton, CT (US)

(21) Appl. No.: 13/809,635

(22) PCT Filed: Jul. 13, 2011
(86) PCT No.: PCT/US11/43785

§ 371 Date: Jan. 11, 2013

Related U.S. Application Data
(60) Provisional application No. 61/364,851, filed on Jul. 16, 2010.

(51) Int. Cl. C12Q 1/02

(52) U.S. Cl. CPC C12Q 1/025
USPC 506/9; 506/10

(57) ABSTRACT

The present invention is based on the surprising finding that pairs of HCV NS5A-targeting inhibitors can be identified which display similar resistance profiles yet, when combined, exhibit synergistic inhibition of wild type replicons and/or replicons carrying mutations conferring resistance to the HCV NS5A-targeting inhibitor. In addition, combinations of these molecules result in a higher genetic barrier to resistance, demonstrating their potential utility as novel combination therapies for treatment of HCV.

### FIELD OF THE INVENTION

The invention relates to a novel experimental strategy for identifying and evaluating HCV NS5A targeting inhibitors that together act synergistically to create a much more potent inhibitory biological response against HCV containing wild-type and/or resistance variants than either single agent can achieve.

### BACKGROUND OF THE INVENTION

Hepatitis C virus (HCV) is the major etiological agent responsible for 90% of all cases of non-A, non-B hepatitis (Dymock, B. W., Emerging Drugs, 6:13 42 (2001)). The incidence of HCV infection is becoming an increasingly severe public health concern worldwide. While primary infection with HCV is often asymptomatic, most HCV infections progress to a chronic state that can persist for decades. Of those with chronic HCV infections, it is believed that about 20-50% will eventually develop chronic liver disease (e.g., cirrhosis) and 20-30% of these cases will lead to liver failure or liver cancer.

Known treatments for HCV infection include the use of interferon-alpha (IFN), which indirectly affects HCV infection by stimulating the host antiviral response. However, IFN treatment is largely ineffective as a sustained antiviral response is produced in less than 30% of treated patients. Further, IFN treatment induces an array of side effects of varying severity in upwards of 90% of patients (e.g., acute pancreatitis, depression, retinopathy, thyroiditis). Therapy with a combination of IFN and ribavirin has provided a higher sustained response rate, but has not alleviated the IFN-induced side effects and can introduce additional side effects, including anemia.

HCV is a positive (+) strand RNA virus which is well characterized, having a length of approximately 9.6 kb and a single, long open reading frame (ORF) encoding an approximately 3000-amino acid polyprotein (Lohman et al., Science, 285:110-113 (1999), expressly incorporated by reference in its entirety). The ORF is flanked at the 5′ end by a non-translated region that functions as an internal ribosome entry site (IRES) and at the 3′ end by a highly conserved sequence essential for genome replication (Lohman, vida supra). The structural proteins are in the amino-terminal region of the polyprotein and the nonstructural proteins (NS) 2 to 5B in the remainder of the protein. Studies have shown that the NS3-5B proteins are all essential for HCV replication and are believed to combine to form the HCV replicase complex.

HCV is a highly heterogeneous virus with resistance variants pre-existing in the viral population in vivo. This is a consequence of the high replication rate of the virus coupled with the lack of proofreading function of the HCV RNA-dependent RNA polymerase. Populations of HCV quasispecies contain greater than one mutation per virus relative to the consensus sequence. Therefore, it can be assumed, at least statistically, that all variants are present in the population and that enrichment of resistance variants may occur during therapy due to selective pressure exerted by the drug (Perelson et al., Science Translational Medicine, 2(30):1 (2010)). Resistance to antiviral therapy has become a major issue in the management of patients with chronic viral infections as the emergence of resistant virus limits the durability of efficacy for small molecules used as monotherapy.

To achieve a sustained viral response in a clinical setting, it will be critical to identify potential combination therapies, especially those comprised of multiple antiviral drugs with different resistance profiles, to suppress the emergence of resistance. The frequency of resistance to a combination of inhibitor molecules is significantly lower than the frequency of resistance to either single inhibitor alone. Combination therapy has most commonly been achieved by targeting different viral proteins or different binding sites on the same viral protein. The use of drug combinations inhibiting distinct HCV viral targets, such as a NS3 protease inhibitor with a NS5B polymerase inhibitor, is known in the art and clinical trials evaluating such combinations are currently underway. Likewise, it is also known that inhibitors binding to different sites on the same viral protein yet showing no cross resistance can be effective inhibitors when used in combination. For example, several structurally distinct classes of non-nucleoside inhibitors have been identified which bind to three different allosteric binding sites on the HCV NS5B polymerase and display non-overlapping resistance profiles. Replicon studies have demonstrated a greater-than-additive inhibitory effect on HCV RNA replication in the presence of combinations targeting two of these distinct sites on the polymerase suggesting that the lack of cross-resistance between these allosteric inhibitors may allow them to be used in combination (Lemm et al., unpublished data).

The present invention is based on the surprising finding that pairs of HCV NS5A targeting inhibitors can be identified which display similar resistance profiles yet when combined exhibit synergistic inhibition of wild type and/or replicons carrying mutations conferring resistance to each individual HCV NS5A targeting inhibitor. In addition, combination of these molecules results in a higher genetic barrier to resistance, demonstrating their potential utility as novel combination therapies for the treatment of HCV.

### SUMMARY OF THE INVENTION

Here we provide a novel approach to identify molecules that can restore the ability of an inhibitor of HCV NS5A to inhibit resistance mutations but which do not act in the traditional fashion by targeting an alternate protein or distinct sites on a protein. Accordingly, herein we describe a method of identifying HCV NS5A-targeting inhibitors that by themselves exhibit inhibitory activity toward viral replication and that, when combined, exert synergistic inhibitory activity toward wild-type replicons and/or replicons harboring mutations that reduce the inhibitory activity of the individual HCV NS5A targeting inhibitors. The claimed method of screening is distinct from screening methods described in the art that identify inhibitory combinations of compounds demonstrating additive or synergistic interactions when said inhibitors target different HCV proteins or target different sites on the same HCV protein, as demonstrated by their non-overlapping resistance profiles. Such combinations excluded from this invention include, for example, HCV NS5A and HCV NS3 inhibitors, HCV NS5A and HCV NS5B inhibitors, HCV NS5A and HCV NS4A inhibitors, HCV NS5A and HCV NS4B inhibitors, HCV NS3 and HCV NS5B inhibitors, HCV NS3 and HCV NS4A inhibitors, HCV NS3 and HCV NS4B inhibitors, HCV NS5B and HCV NS4A inhibitors, HCV NS5B and HCV NS4B inhibitors and two HCV NS5B inhibitors that act at different sites of the enzyme.

### BRIEF DESCRIPTION OF THE DRAWINGS

FIG. 1 shows the suppression of hyperphosphorylation (p58) of GT1b NS5A with Compound A and Compound B. GT 1b Y93H NS5A was expressed using the vaccinia expression system in the presence or absence of compounds. p56 and p58 were detected by Western blot.

FIG. 2 shows the suppression of hyperphosphorylation (p58) of GT1a NS5A with Compound C and Compound D. GT 1a wild type NS5A was expressed using the vaccinia expression system in the presence or absence of compounds. p56 and p58 were detected by Western blot.

FIG. 3 shows a colony formation assay in GT 1a wild type replicon treated with 20 nM Compound E, 10 nM Compound F or a combination thereof

### DETAILED DESCRIPTION OF THE INVENTION

The present invention provides a method for identifying combinations of HCV NS5A-targeting compounds that together act synergistically to create a much more potent inhibitory biological response toward HCV than either single agent alone can achieve. The method comprises: (a) determining the amount of HCV inhibition by an NS5A targeting compound and (b) comparing the amount of HCV inhibition of said NS5A-targeting compound in the presence and absence of a fixed concentration of a second NS5A-targeting compound.

The assay strategy of the present invention identifies combinations of molecules with potent anti-HCV properties and maximizes the potential to detect active compounds in a library by screening a library of NS5A inhibitors in the presence of one or more primary NS5A-targeting compounds. The library compounds themselves typically demonstrate antiviral activity and, when used in combination, enhance in a synergistic fashion the potency of the NS5A-targeting inhibitor, particularly towards HCV sequences incorporating one or more substitutions in NS5A that confer resistance to the primary inhibitor.

The assay strategy of the present invention includes a cell-based HCV assay for measuring the ability of compounds to interact synergistically. Preferably, an assay of the present invention includes the use of cells transfected with a HCV replicon, including replicon cell lines. Accordingly, the HCV replicon systems utilized in the assay strategy of the invention include but are not limited to 1) genotype (GT) 1b replicons carrying different single amino acid substitutions (L31V, Y93H) in NS5A; 2) a genotype 1b replicon carrying two amino acid substitutions (L31V and Y93H) in NS5A; 3) a GT 1a wild type (WT) replicon; 4) GT 1a replicons carrying different single amino acid substitutions (M28T, Q30R, Q30H, Q30E, L31V, Y93H, Y93N) in NS5A; 5) GT 1a resistant replicons carrying two amino acid substitutions (L31V and Y93H, M28T and Q30H, Q30R and H58D, Q30H and Y93H, Q30R and E62D) in NS5A and combinations thereof; 6) a GT 2a WT replicon and variants thereof; 7) a GT 3a WT replicon and variants thereof.

Preferably, the assay strategy of the present invention utilizes luciferase or other reporter enzymes (such as beta-lactamase) or indicators (such as green fluorescence protein) and/or qRT/PCR and/or fluorescence resonance energy transfer (FRET)-based methods (O'Boyle et al., Antimicrob. Agents Chemother. 49:1346-1353 (2005)). Alternative methods of detecting synergistic compound inhibitory effects include, but are not limited to, assays relying on Western analysis, an assessment of NS5A hyperphosphorylation, and/or colony formation. The assay strategy of the present invention is amenable to high-throughput screening (HTS) to identify combinations of two or more HCV NS5A-targeting inhibitors that interact synergistically to inhibit HCV RNA replication, providing a convenient and economical strategy to maximize the potential to identify compound combinations from a particular library of compounds.

### DEFINITIONS

The term resistance variant means an HCV sequence containing substitutions in NS5A that reduce the susceptibility to HCV NS5A-targeting inhibitors. Resistance variants include, but are not limited to, genotype 1b sequence carrying a Y93H single amino acid substitution in NS5A, genotype 1b sequence carrying a L31V single amino acid substitution in NS5A, genotype 1b sequence carrying amino acid substitutions at both L31V and Y93H in NS5A, genotype 1a sequence carrying a M28T single amino acid substitution in NS5A, genotype 1a sequence carrying a Q30R single amino acid substitution in NS5A, genotype 1a sequence carrying a L31V single amino acid substitution in NS5A, genotype 1a sequence carrying a Y93H single amino acid substitution in NS5A, genotype 1a sequence carrying a Q30H single amino acid substitution in NS5A, genotype 1a sequence carrying a Y93N single amino acid substitution in NS5A, genotype 1a sequence carrying a Q30E single amino acid substitution in NS5A, genotype 1a sequence carrying amino acid substitutions at both L31V and Y93H in NS5A, genotype 1a sequence carrying amino acid substitutions at both M28T and Q30H in NS5A, genotype 1a sequence carrying amino acid substitutions at both Q30R and H58D in NS5A, genotype 1a sequence carrying amino acid substitutions at both Q30H and Y93H in NS5A, genotype 1a sequence carrying amino acid substitutions at both Q30R and E62D in NS5A and other combinations beyond those listed here that may arise in response to selective pressure exerted by HCV NS5A-targeting compounds.

Cell-based method is defined as an assay for measuring inhibitory activity against HCV or HCV derived replicons in tissue culture cells and includes, but is not limited to, a FRET assay, luciferase assay, qRT-PCR assay, Western blot analysis, ELISA, Northern analysis and colony formation assay.

Biochemical surrogate refers to measuring phosphorylation levels of HCV NS5A and includes, but is not limited to, using a vaccinia expression system or replicon cells.

Synergy is defined as the interaction of two or more agents such that their combined effect is greater than the sum of their individual effects. Preferably, synergy refers to a greater than or equal to 3-fold enhancement in anti-HCV inhibitory effect resulting from combination of two NS5A targeting compounds. Examples of NS5A targeting compounds utilized to demonstrate the claimed method include but are not limited to Compound A, Compound B, Compound C, Compound D, Compound E, Compound F, Compound G, Compound H, and Compound I.

Compounds F, H and I are described in co-pending application WO2008/021927, which is expressly incorporated herein by reference in its entirety and describes compounds of Formula (I)

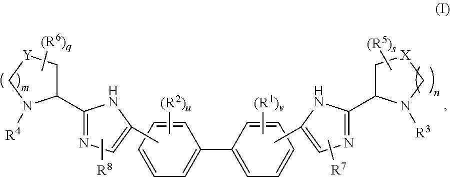

or a pharmaceutically acceptable salt thereof, wherein

m and n are independently 0, 1, or 2;

q and s are independently 0, 1, 2, 3, or 4;

u and v are independently 0, 1, 2, or 3;

X is selected from O, S, S(O), SO₂, CH₂, CHR⁵, and C(R⁵)₂;

provided that when m is O, X is selected from CH₂, CHR⁵, and C(R⁵)₂;

Y is selected from O, S, S(O), SO₂, CH₂, CHR⁶, and C(R⁶)₂;

provided that when n is O, Y is selected from CH₂, CHR⁶, and C(R⁶)₂;

each R¹ and R² are each independently selected from alkoxy, alkoxycarbonyl, alkyl, carboxy, halo, haloalkyl, hydroxy, —NR^{a}R^{b}, (NR^{a}R^{b})alkyl, and (NR^{a}R^{b})carbonyl;

R³ and R⁴ are each independently selected from hydrogen and R⁹—C(O)—;

each R⁵ and R⁶ is independently selected from alkoxy, alkyl, halo, haloalkyl, hydroxy, and —NR^{a}R^{b}, wherein the alkyl can optionally form a fused cyclopropyl ring with an adjacent carbon atom;

R⁷ and R⁸ are each independently selected from hydrogen, alkoxycarbonyl, alkyl, carboxy, haloalkyl, (NR^{a}R^{b})carbonyl, and trialkylsilylalkoxyalkyl; and

each R⁹ is independently selected from alkoxy, alkoxyalkyl, alkoxycarbonyl, alkoxycarbonylalkyl, alkyl, alkylcarbonylalkyl, aryl, arylalkenyl, arylalkoxy, arylalkyl, aryloxyalkyl, cycloalkyl, (cycloalkyl)alkenyl, (cycloalkyl)alkyl, cycloalkyloxyalkyl, haloalkyl, heterocyclyl, heterocyclylalkenyl, heterocyclylalkoxy, heterocyclylalkyl, heterocyclyloxyalkyl, hydroxyalkyl, —NR^{c}R^{d}, (NR^{c}R^{d})alkyl, and (NR^{c}R^{d})carbonyl.

Compound G is described in co-pending application WO2009/102318, which is expressly incorporated herein by reference in its entirety and describes compounds of Formula (II)

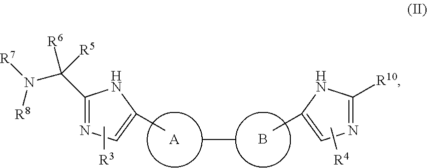

or a pharmaceutically acceptable salt thereof, wherein

A and B are independently selected from phenyl and a six-membered heteroaromatic ring containing one, two, or three nitrogen atoms;

R³ and R⁴ are each independently selected from hydrogen, haloalkyl, and trialkylsilylalkoxyalkyl;

R⁵ and R⁶ are each independently selected from hydrogen, and alkyl;

R⁷ is selected from hydrogen and R⁹—C(O)—;

R⁸ is selected from hydrogen and alkyl;

R⁹ is independently selected from alkoxy, arylalkoxy, arylalkyl, and (NR^{c}R^{d})alkyl;

R¹⁰ is selected from

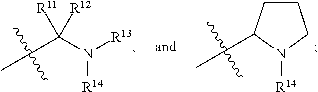

wherein

R¹¹ and R¹² are each independently selected from hydrogen and alkyl;

R¹³ is selected from hydrogen and alkyl;

R¹⁴ is selected from hydrogen and R¹⁵—C(O)—; and

R¹⁵ is independently selected from alkoxy, arylalkoxy, arylalkyl, and (NR^{c}R^{d})alkyl.

Compounds A, B, C and D are described in co-pending application WO2006/133326, which is expressly incorporated herein by reference in its entirety and describes compounds of formula (III)

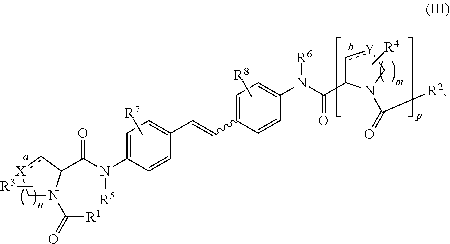

or pharmaceutically acceptable salts thereof, wherein

[symbol] is a single or double bond;

[symbol] is a single or double bond;

when [symbol] is a single bond, X is selected from the group consisting of O, CH₂, and CHR³;

when [symbol] is a double bond, X is selected from the group consisting of CH and CR³;

when [symbol] is a single bond, Y is selected from the group consisting of O, CH₂, and CHR⁴;

when [symbol] is a double bond, Y is selected from the group consisting of CH and CR⁴;

n and m are independently 0, 1, 2, or 3;

p is 0 or 1;

R¹ and R² are independently selected from the group consisting of alkoxy, alkoxyalkoxyalkyl, alkoxyalkyl, alkyl, alkylsulfenylalkyl, alkylsulfinylalkyl, alkylsulfonylalkyl, aryl, arylalkoxy, arylalkoxyalkyl, arylalkyl, arylcarbonyl, aryloxy, aryloxyalkyl, arylsulfenylalkyl, arylsulfinylalkyl, arylsulfonylalkyl, carboxyalkyl, cycloalkenyl, cycloalkenylalkyl, cycloalkyl, cycloalkylalkyl, heterocyclyl, heterocyclylalkoxy, heterocyclylalkoxyalkyl, heterocyclylalkyl, heterocyclylcarbonyl, heterocyclyloxy, heterocyclyloxyalkyl, —NR^{a}R^{b}, and (NR^{a}R^{b})alkyl;

R³ and R⁴ are independently selected from the group consisting of hydrogen, alkenyl, alkoxy, alkoxycarbonyloxy, alkyl, alkylsulfonyl, alkylsulfonyloxy, aryl, arylalkyl, azido, hydroxy, —NR^{a}R^{b}, (NR^{a}R^{b})alkyl, and (NR^{a}R^{b})carbonyloxy; wherein the alkenyl and the alkyl can optionally form a saturated or unsaturated cyclic structure, respectively, with an adjacent carbon atom;

R⁵ and R⁶ are independently selected from the group consisting of hydrogen, alkenyl, alkoxycarbonyl, alkyl, alkylcarbonyl, aryl, arylalkyl, heterocyclylalkylcarbonyl, and heterocyclylcarbonyl;

R⁷ and R⁸ are independently selected from the group consisting of hydrogen, alkenyl, alkoxy, alkyl, halo, and haloalkyl; and

R^{a} and R^{b} are independently selected from the group consisting of hydrogen, alkenyl, alkyl, alkylcarbonyl, aryl, arylalkyl, arylalkylcarbonyl, cycloalkyl, cycloalkylalkyl, heterocyclyl, and heterocyclylalkyl.

Compound E can be manufactured by the methods described in co-pending application WO2010/062821, which is expressly incorporated herein by reference in its entirety.

The following preparations and examples are given to enable those skilled in the art to more clearly understand and to practice the present invention. They should not be considered as limiting the scope of the invention, but merely as being illustrative and representative thereof Numerous changes, modifications and alterations can be employed without departing from the spirit and scope of the invention.

### Materials and Methods

### HCV Replicon Cell Lines

The HCV replicon cell lines were isolated from colonies as described by Lohman et al. (Science, 285:110-113 (1999)), which is expressly incorporated herein by reference in its entirety. HCV replicon cell lines were maintained at 37° C. in Dulbecco's modified Eagle medium (11965-084; Life Technologies) with 10% heat inactivated calf serum (Sigma), penicillin-streptomycin (Life Technologies) and 1 mg/ml GENETICIN® (Life Technologies).

The HCV replicon systems utilized to exemplify the assay strategy of the invention include: 1) genotype (GT) 1b replicons carrying different single amino acid substitutions (L31V, Y93H) in NS5A; 2) a genotype 1b replicon carrying two amino acid substitutions (L31V and Y93H) in NS5A; 3) a GT 1a wild type (WT) replicon; 4) GT 1a replicons carrying different single amino acid resistance substitutions (M28T, Q30E, Q30H, Q30R, L31V, Y93H, Y93N) in NS5A; 5) GT 1a resistant replicons carrying two amino acid substitutions (M28T-Q30H, Q30H-Y93H, Q30R-E62D, L31V-Y93H) in NS5A and combinations thereof; 6) a GT 2a WT replicon; 7) a GT 3a WT replicon. Methods to select and isolate these HCV resistance replicon cell lines as well as assay conditions were described previously (Lemm et al., J. Virology 84:482-491, 2010; Gao et al., Nature, 465:96-100 (2010); Fridell et al., Antimicrob. Agents and Chemother., in press (2010)).

Other HCV replicons, as well as different genotypes, are suitable for use in the assay strategy of the present invention, and it is to be understood that the assay strategy of the present invention is not limited to any particular HCV replicon or cell line created therefrom. Also, it is understood that modifications of such HCV replicons may be made such that the replicon is useful in the assay strategy of the present invention.

### HCV EC₅₀ Determination

The ability of a pair of NS5A-targeting inhibitors to synergistically inhibit HCV was assessed by determining the EC₅₀ values for an individual NS5A-targeting inhibitor in the presence and absence of a given concentration of a second NS5A-targeting compound. HCV replicon cells were seeded in 96-well plates in DMEM containing 10% FBS at a cell density of 10⁴/well and incubated at 37° C. overnight. NS5A-targeting compounds were serially diluted in DMSO and added to the cell plates in the presence or absence of various fixed concentrations of a second NS5A-targeting inhibitor. The plates were then incubated at 37° C. for three days and the amount of HCV inhibition generated by the single NS5A-targeting compound was compared to that produced by the combination of NS5A-targeting inhibitors.

For luciferase reporter replicons, inhibition of HCV was assessed by measuring renilla luciferase activity using a Renilla Luciferase Assay System (Promega Corporation, Madison, Wis.) according to the manufacturer's directions. Plates were read on a TOPCOUNT® NXT Microplate Scintillation and Luminescence Counter (Packard Instrument Company, Meriden Conn.). For replicons lacking a reporter gene, NS3 protease activity was used as an indirect measure of the amount of HCV replicon RNA present within cells. NS3 protease activity was measured using a FRET assay, as described previously (O'Boyle et al., Antimicrob. Agents Chemother., 49:1346-1353 (2005)), which is expressly incorporated herein by reference in its entirety, and plates read on a CYTOFLUOR® 4000 instrument (340 nm excitation/490 nm emission). Linear rates obtained up to 20 cycles in kinetic mode were used in EC₅₀ calculations. The 50% effective concentration (EC₅₀) represents the inhibitor concentration that yields a RNA value halfway between baseline and maximum. In cases where inhibition was observed by the fixed concentration of NS5A compound alone, the EC₅₀ values of the combination were determined after subtraction of the percent inhibition generated by the fixed concentration alone.

### NS5A Hyperphosphorylation Assays

Mammalian transient expression assays using the vaccinia-T7 hybrid system were performed as described previously (Lemm, et al., J. Virol., 84:482-491 (2010); Fridell et al., Antimicrob. Agents and Chemother., in press (2010)), and are expressly incorporated herein by reference in its entirety. Briefly, monolayers of BHK-21 cells were infected with vTF7-3 at a multiplicity of infection of 1 plaque forming unit per cell for 1 h at room temperature. After removal of the inoculum, cells were transfected with a mixture of plasmid DNA, plus reagent and lipofectamine (Invitrogen) according to the manufacturer's directions and incubated in the absence or presence of compound for 7 h. To detect p56 and p58 by Western analysis, transfected cells were lysed using cell dissociation buffer and material from equal numbers of cells was separated on an 8% acrylamide gel by SDS-PAGE. After electrophoretic transfer, the HCV NS5A protein was detected with rabbit antiserum specific for NS5A and secondary goat anti-rabbit horseradish peroxidase-conjugated antibody followed by the ECL detection system (Amersham Biosciences).

### NS5A Colony Formation Assays

The colony formation assay was conducted by placing HCV replicon cells into cell culture dishes at a density required to obtain a confluent monolayer at the end of the exposure period; typically 24,000 cells per 100 mm dish. Compound(s) at differing concentrations were then placed into DMEM with or without 1 mg/mL GENETICIN® (G418) and added to the plated cells. The cells/media/compounds were placed in an incubator for the desired period of exposure, typically 7 days. Following exposure to inhibitors, the medium was removed, the cells were washed 2× with DMEM containing 1 mg/mL G418, and incubation was continued until distinct colonies were visible or a complete cell monolayer was obtained, typically 14 days. The cells were then stained with a crystal violet solution and photographed. HCV replicon cells which were inhibited by a treatment no longer produced resistance to the amino-glycoside antibiotic G418 and were removed from the dishes resulting in no visible staining.

### Synergistic Inhibition of HCV Replicons by Combinations of HCV NS5A-Targeting Compounds

To identify NS5A-targeting inhibitors that, in combination, displayed synergistic inhibition of HCV, the EC₅₀ values for a specific NS5A-targeting inhibitor were determined in the presence and absence of a given concentration of a second NS5A-targeting compound. In this assay, for a specific strain of interest, at each increasing concentration of a given NS5A-targeting inhibitor (e.g., Compound F), the potency of the test compound is determined A synergistic inhibitory effect occurs when the potency of the two compounds combined (e.g., Compound F and the test compound) is more than the sum of potency of the individual compounds when tested alone. An example of a pair of NS5A-targeting compounds discovered by this screening strategy that demonstrate a synergistic inhibitory effect is Compound F and Compound G. Compound F is a highly potent inhibitor of GT 1b wild-type replicon and resistant variants carrying single amino acid substitutions in NS5A (pM range) (Gao et al., Nature, 465:96-100 (2010)); however, highly resistant variants carrying two amino acid substitutions, such as the GT 1b L31V-Y93H variant which has substitutions at residues L31 and Y93 in NS5A exist. The EC₅₀ values for Compound F on the wild-type and L31V-Y93H GT 1b replicons are 0.009 nM and ˜400 nM, respectively, while the EC₅₀ values for Compound G are ˜300 nM and >5,000 nM on GT 1b wild-type and L31V-Y93H resistance replicons, respectively. In this experiment, the EC₅₀ of Compound G toward the GT 1b L31V-Y93H variant was >1,000 nM in the absence of Compound F (Table 1, left panel, 0 nM Compound F). However, when Compound G was titrated in the presence of 40 nM Compound F, the EC₅₀ of Compound G was synergistically enhanced from >1,000 nM to 133 nM, even though only approximately 5% inhibition of the GT 1b L31V-Y93H variant was observed with 40 nM Compound F alone (Table 1, left panel).

TABLE 1
Synergistic Inhibition of the GT 1b L31V-Y93H Replicon
by a Combination of Compound F and Compound G

| GT 1b L31V-Y93H replicon | | | GT 1b L31V-Y93H replicon | | |
|---|---|---|---|---|---|
| nM of | Percent inhib. | EC₅₀ (nM) of | nM of | Percent inhib. | EC₅₀ (nM) of |
| Compound F^{a} | by Compound F^{b} | Compound G | Compound G^{c} | by Compound G^{d} | Compound F |
| 0 | 0 | >1,000 | 0 | 0 | 435 |
| 1.6 | −4 | >1,000 | 8 | 9 | 315 |
| 8 | 2 | 418 | 40 | 14 | 194 |
| 40 | 5 | 133 | 200 | 15 | 38 |
| 200 | 21 | 32 | 1,000 | 23 | 2.5 |

^{a}Concentration of Compound F included in the Compound G titration.
^{b}Percent inhibition of HCV at various concentrations of Compound F alone.
^{c}Concentration of Compound G included in the Compound F titration.
^{d}Percent inhibition of HCV at various concentrations of Compound G alone.

Similarly, when Compound G was titrated in the presence of 200 nM Compound F, the EC₅₀ of Compound G was synergistically enhanced from >1,000 nM to 32 nM (Table 1, left panel). In the reciprocal experiment, the EC₅₀ value of Compound F on the GT 1b L31V-Y93H variant was synergistically enhanced from 435 nM to 2.5 nM in the presence of 1,000 nM Compound G (Table 1, right panel).

To demonstrate the broad utility of this methodology, synergistic inhibitory effects were evaluated using additional resistance mutants including the GT 1a Y93H replicon (Table 2). The EC₅₀ values of Compound F for the GT 1a wild-type and Y93H variant were ˜50 pM and 40-190 nM, respectively, while the EC₅₀ values of Compound G were >1,000 nM for both wild-type and the Y93H variant. In this experiment, the EC₅₀ of Compound G on the GT 1a Y93H variant was >1,000 nM in the absence of Compound F (Table 2, left panel). Approximately 3% inhibition of the GT 1a Y93H variant was observed by 1.6 nM Compound F (Table 2, left panel). However, when Compound G was titrated in the presence of 1.6 nM Compound F, the EC₅₀ of Compound G was synergistically enhanced from >1,000 nM to 3.2 nM. In the reciprocal experiment, the EC₅₀ value of Compound F on the GT 1a Y93H variant was synergistically enhanced from 17 nM to 0.046 nM in the presence of 200 nM Compound G (Table 2, right panel).

TABLE 2
Synergistic Inhibition of the GT 1a Y93H Replicon
by a Combination of Compound F and Compound G

| GT 1a Y93H replicon | | | GT 1a Y93H replicon | | |
|---|---|---|---|---|---|
| nM of | Percent inhib. | EC₅₀ (nM) of | nM of | Percent inhib. | EC₅₀ (nM) of |
| Compound F | by Compound F | Compound G | Compound G | by Compound G | Compound F |
| 0 | 0 | >1,000 | 0 | 0 | 17 |
| 0.0026 | 3 | 698 | 1.6 | 3 | 3.6 |
| 0.064 | 9 | 77 | 8 | 4 | 0.74 |
| 0.32 | 0 | 21 | 40 | 24 | 0.42 |
| 1.6 | 3 | 3.2 | 200 | 18 | 0.046 |

The synergistic inhibitory effect was also evaluated in the GT 1a Q30E replicon (Table 3). The EC₅₀ values of Compound F for the GT 1a wild-type and Q30E variant were ˜50 pM and ˜210 nM, respectively, while the EC₅₀ values of Compound G were >1,000 nM for both the wild-type and Q30E variant. In this synergy experiment, the EC₅₀ of Compound G on the GT 1a Q30E variant was >1,000 nM in the absence of Compound F (Table 3, left panel). When Compound G was titrated in the presence of 8 nM Compound F, the EC₅₀ of Compound G was synergistically enhanced from >1,000 nM to 57 nM.

TABLE 3
Synergistic Inhibition of the GT 1a Q30E Replicon
by a Combination of Compound F and Compound G

| GT 1a Q30E | | | GT 1a Q30E | | |
|---|---|---|---|---|---|
| nM of | Percent inhib. | E₅ (nM) of | nM of | Percent inhib. | E₅ (nM) of |
| Compound F | by Compound F | Compound G | Compound G | by Compound G | Compound F |
| 0 | 18 | >1,00 | 0 | 18 | 185 |
| 0.06 | 11 | >1,00 | 1. | −2 | 96 |
| 0.3 | 13 | 917 | 8 | 11 | 46 |
| 1. | 22 | 197 | 40 | 5 | 11 |
| 8 | 15 | 57 | 200 | 7 | 1.5 |

In the reciprocal experiment, the EC₅₀ value of Compound F on the GT 1a Q30E variant was synergistically enhanced from 185 nM to 1.5 nM in the presence of 200 nM of Compound G (Table 3, right panel).

The synergistic inhibitory effect was also evaluated in the GT 1a Q30R-E62D replicon (Table 4). This variant carries two amino acid substitutions at residues Q30 and E62 in NS5A. The EC₅₀ values of Compound F for the GT 1a wild-type and Q30R-E62D variant were ˜50 pM and ˜150 nM, respectively, while the EC₅₀ values of Compound G were >1,000 nM for both the wild-type and Q30R-E62D variant. In the synergy experiment, the EC₅₀ of Compound G on the GT 1a Q30R-E62D variant was >1,000 nM in the absence of Compound F (Table 4, left panel). When Compound G was titrated in the presence of 8 nM Compound F, the EC₅₀ of Compound G was synergistically enhanced from >1,000 nM to 18 nM. In the reciprocal experiment, the EC₅₀ value of Compound F on the GT 1a Q30R-E62D variant was enhanced from 181 nM to 0.37 nM in the presence of 200 nM of Compound G (Table 4, right panel).

TABLE 4
Synergistic Inhibition of the GT 1a Q30R-E62D Replicon
by a Combination of Compound F and Compound G

| GT 1a Q30R-E62D replicon | | | GT 1a Q30R-E62D replicon | | |
|---|---|---|---|---|---|
| nM of | Percent inhib. | EC₅₀ (nM) of | nM of | Percent inhib. | EC₅₀ (nM) of |
| Compound F | by Compound F | Compound G | Compound G | by Compound G | Compound F |
| 0 | 0 | >1,000 | 0 | 0 | 181 |
| 0.32 | 1 | >1,000 | 40 | 11 | 1.4 |
| 8 | 4 | 18 | 200 | 4 | 0.37 |

In addition to the HCV replicon cell lines discussed above, additional replicon cell lines were examined for their ability to expose synergistic inhibitory activities, and the data is summarized in Table 5. Since Compound F is already a very potent inhibitor (in the pM range) of 1a wild-type and 1b resistance variants carrying single amino acid substitutions (such as GT 1b L31V, Y93H), evaluation of the synergistic inhibitory effect is focused more extensively on GT 1a resistance variants. As shown in Table 5, synergistic inhibition was observed for a variety of resistant variants carrying both single and double amino acid substitutions. For instance, the EC₅₀ values of Compound G and Compound F were >1,000 nM and 1,400 nM, respectively for the GT 1a M28T-Q30H variant (carrying two amino acid substitutions) (Table 5), respectively. In this synergy experiment, minimal inhibition (−10%).of the M28T-Q30H variant was observed in the presence of 300 nM of Compound F.

TABLE 5
Summary of Synergistic Inhibitory Effect of HCV NS5A Targeting Inhibitors
(Compound F and Compound G) on Multiple HCV Replicon Cell Lines

| | | | EC₅₀ (nM) of | | Percent | |
|---|---|---|---|---|---|---|
| | | | Compound G in the | | inhibition | |
| | EC₅₀ (nM) | EC₅₀ (nM) | presence of (x nM)^{a} | | by (x nM)^{b} | |
| Replicon cell lines | Compound G | Compound F | Compound F | | Compound F | |
| 1a-WT | >1,000 | 0.05 | 0.5 | (0.013) | 15 | (0.013) |
| 1a-M28T | >1,000 | 10 | 0.4 | (11) | 29 | (11) |
| 1a-Q30H | >1,000 | 9 | 0.5 | (6.4) | 19 | (6.4) |
| 1a-Q30R | >1,000 | 9 | 0.85 | (14) | 46 | (14) |
| 1a-L31V | >1,000 | 39 | 1.1 | (40) | 65 | (40) |
| 1a-Y93N | >1,000 | 581 | 2 | (300) | 60 | (300) |
| 1a-M28T-Q30H | >1,000 | 1400 | 11 | (300) | −10 | (300) |
| 1a-Q30H-Y93H | >1,000 | 380 | 3 | (222) | 54 | (222) |
| 1a-L31V-Y93H | 947 | 8100 | 124 | (300) | 1 | (300) |
| 1b-L31V | >1,000 | 0.18 | 22 | (0.081) | 45 | (0.081) |
| 1b-Y93H | >1,000 | 0.55 | 73 | (0.39) | 60 | (0.39) |

^{a}The number in parenthesis indicates the concentration (in nM) of Compound F included in the Compound G titration.
^{b}The number in parenthesis indicates the concentration (in nM) of Compound F tested alone.

However, when Compound G and Compound F were combined, the EC₅₀ of Compound G was synergistically enhanced from >1,000 nM to 11 nM in the presence of 300 nM of Compound F (Table 5).

### Synergistic Combinations of HCV NS5A-Targeting Compounds Broaden Genotype Coverage

In addition to dramatically enhancing the potency of HCV NS5A-targeting compounds against resistance variants, combinations of HCV NS5A-targeting inhibitors were also observed to demonstrate synergistic inhibitory activity toward different genotype HCV replicons.

TABLE 6
EC₅₀s of Compound H and Compound
I on GT 2 and 3 Replicon Cells

| Replicon lines | Compound H | Compound I (nM) |
|---|---|---|
| GT 2a (HC-J6CF) | 13.6 | 308 |
| GT 2a (JFH) | 0.033 | 4.4 |
| GT 3a | 0.64 | 39 |
| GT 3a Y93H | 23 | 116 |

The EC₅₀ values of Compound H and Compound I on GT 2 and 3 replicon cells are listed in Table 6. In the synergy combination experiments, the EC₅₀ of Compound H was 17 nM by itself in the GT 2a strain HC-J6CF, but the potency was enhanced to 0.89 nM in the presence of 150 nM of Compound I (Table 6-a).

TABLE 6-a

| | Compound I | % inhibition | EC50 of |
|---|---|---|---|
| | (nM) | by Compound I | Compound H |
| 2a-HC-J6CF | 0 | 0 | 17 |
| | 37.5 | 29 | 7.7 |
| | 75 | 36 | 3.7 |
| | 150 | 54 | 0.89 |

TABLE 6-b

| | Com I | % inhibition | EC50 of |
|---|---|---|---|
| | (nM | by Compound | Comp H |
| 2a- | 0 | 0 | 0.032 |
| | 1.25 | 15 | 0.004 |
| | 2.5 | 26 | 0.003 |
| | 5 | 70 | 0.011 |

TABLE 6-c

| | Compound-I | % inhibition | EC50 of |
|---|---|---|---|
| | (nM | by Compound I | Compound-H |
| 3 | 0 | 0 | 0.73 |
| | 1.25 | 28 | 0.0022 |
| | 2.5 | 30 | 0.0011 |
| | 5 | 23 | 0.0008 |

TABLE 6-d

| | Compound I | % inhibition | EC50 of |
|---|---|---|---|
| | (nM | by Compound I | Compound H |
| 3a- | 0 | 0 | 24 |
| | 3.7000 | 22 | 0.30 |
| | 11.0000 | 24 | 0.10 |
| | 33.0000 | 31 | 0.05 |
| | 100 | 60 | 0.026 |

Similarly, in the presence of Compound I, the potency of Compound H was markedly enhanced against the GT 2a JFH strain (Table 6-b) and a GT 3a wild type (Table 6-c) and Y93H resistant (Table 6-d) replicon cells. These results demonstrate that combinations of NS5A-targeting compounds can synergistically enhance potency against genotypes other than GT1a and GT1b, such as GT 2 and 3, thereby broadening the genotype coverage of the primary inhibitor.

Additional methods were utilized to further validate the experimental strategy of identifying combinations of NS5A-targeting compounds that demonstrate synergistic inhibitory effects. Examples of two such methods, an NS5A hyperphosphorylation assay and colony formation assay, are detailed below.

Suppression of NS5A Hyperphosphorylation Correlates with Synergistic Inhibition

NS5A is known to be a phosphoprotein, with basally phosphorylated (p56) and hyperphosphorylated (p58) forms (Kaneko et al., Biochem. Biophys. Res. Commun., 205:320-326 (1994); Neddermann et al., J. Virol., 73:9984-9991 (1999)). Previously, a functional assay was developed to determine the impact of inhibitors on NS5A hyperphosphorylation (Lemm et al., J. Virol., 84:482-491 (2010)), which is expressly incorporated herein by reference in its entirety. Briefly, NS5A inhibitors were evaluated for their ability to block p58 formation in a vaccinia system expressing WT NS5A, either from the HCV NS3-NS5B or NS3-NS5A polyprotein (Lemm et al., J. Virol., 84:482-491 (2010)). The concentration of a NS5A inhibitor required for 50% inhibition of HCV replication (EC₅₀) correlates well with the concentration required to block p58 formation (Lemm et al., J. Virol., 84:482-491 (2010)). This functional assay was used to determine how combinations of NS5A-targeting compounds that produce synergistic inhibition of HCV replication impact NS5A phosphorylation. The synergistic inhibitory effects of Compound B and Compound A were quantified in the replicon assay, as shown in Table 7. The EC₅₀ values of Compound A and Compound B alone in the GT 1b Y93H replicon are 662 and >10,000 nM, respectively (Table 7). In the presence of 200 nM Compound B, the EC₅₀ of Compound A on the Y93H variant was synergistically enhanced from 662 nM to <14 nM (Table 7, left panel, 93% inhibition). In the reciprocal experiment, the EC₅₀ of Compound B on the Y93H variant was synergistically enhanced from >10,000 nM to 34 nM (Table 7, right panel).

TABLE 7
Synergistic Inhibition of the GT 1b Y93H Replicon by Compound B and Compound A

| nM of | % of Y93H inhibited | EC₅₀ (nM) of | nM of | % of Y93H inhibited | EC₅₀ (nM) of |
|---|---|---|---|---|---|
| Comp-B | by Compound B | Compound A | Comp A | by Compound A | Compound B |
| 0 | 0 | 662 | 0 | 0 | >10,000 |
| 200 | 0 | <14 (93% inhi.) | 40 | — | 53 |
| | | | 100 | — | 34 |

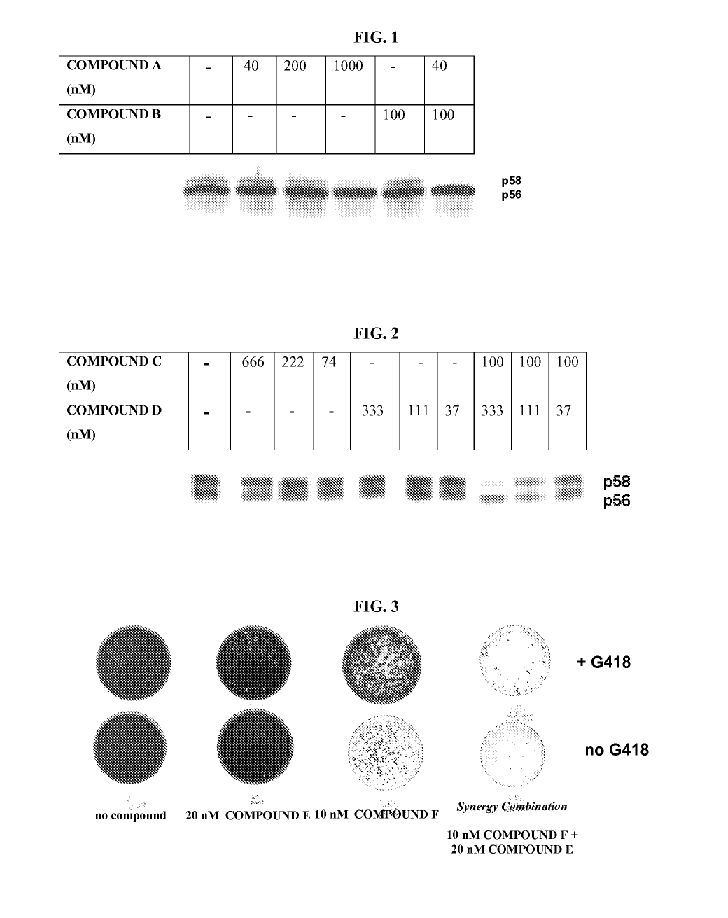

Suppression of NS5A hyperphosphorylation was evaluated in parallel in the vaccinia system. No suppression of p58 formation was observed with up to 200 nM Compound A or 100 nM Compound B (FIG. 1). However, when a GT 1b Y93H replicon was treated with a combination of 40 nM Compound A and 100 nM Compound B, the formation of p58 was completely blocked (FIG. 1, lane 6), demonstrating a direct correlation between the synergistic inhibition of HCV RNA replication and suppression of p58 formation.

Suppression of NS5A hyperphosphorylation was also evaluated in GT 1a wild type replicon (FIG. 2). The EC₅₀ values of Compound C and Compound D were 460 nM and 340 nM, respectively in the experiments performed in Table 8. The EC₅₀ values of Compound C were enhanced from 460 nM in the absence of Compound D to 14 nM in the presence of 170 nM Compound D (Table 8, left panel). Similarly, the EC₅₀ values of Compound D were enhanced from 340 nM in the absence of Compound C to 14 nM in the presence of Compound C (170 nM, Table 8, right panel). When a GT 1a replicon was treated with a combination of 100 nM Compound C and 333 nM Compound D, the formation of p58 was completely blocked (FIG. 2, lane 8), demonstrating a direct correlation between the synergistic inhibition of HCV RNA replication and suppression of p58 formation in wild type GT1a.

TABLE 8
Synergic Inhibition of WT GT 1a Replicon with Compound D and Compound C

| | % of GT 1a WT | | | % of GT 1a WT | |
|---|---|---|---|---|---|
| Compound D | inhibited by | Compound C | Compound C | inhibited by | Compound D |
| nM | Compound D | EC50 (nM) | nM | Compound C | EC50 (nM) |
| 0 | −4 | 460 | 0 | 1 | 340 |
| 55 | 26 | 40 | 55 | 27 | 24 |
| 170 | 27 | 14 | 170 | 25 | 14 |

### Increasing Resistance Barriers by Synergistic Inhibition

A colony formation assay was used to determine whether a combination of two NS5A-targeting inhibitors that exhibit synergistic inhibition was more effective at eliminating HCV replicon from cells than treatment with the individual compounds, thereby increasing the genetic barrier for resistance development.

The EC₅₀ values of Compound F on GT 1a L31V and Y93H replicons were 38 nM and 130 nM, respectively, while the EC₅₀ values of Compound E on GT 1a L31V and Y93H replicons were >200 nM (Table 9). However, in the presence of 200 nM Compound E, the EC₅₀ values of Compound F were synergistically enhanced to 0.38 nM for the L31V resistance variant and 0.06 nM for the Y93H resistance variant. Similarly, in the presence of 33 nM Compound F, the EC₅₀ values of Compound E were synergistically enhanced to 1 nM for the L31V resistance variant and 0.35 nM for the Y93H resistance variant.

TABLE 9
Synergistic Inhibition of GT 1a Resistant Replicons by a Combination of Compound F and Compound E

| | | | EC₅₀ (nM) Comp. F in the | | EC₅₀ (nM) Comp E in the | |
|---|---|---|---|---|---|---|
| | EC₅₀ | | presence of 200 nM | Enhancement (fold | presence of 33 nM | Enhancement (fold |
| GT | — | — | of Compound E | change) | of Comp. F | change) |
| WT | 0.0 | >12 | | | | |
| L31 | 3 | >20 | 0.3 | 10 | 1 | >20 |
| Y93 | 13 | >20 | 0.0 | 316 | 0.3 | >57 |

To determine whether this synergistic enhancement of potency impacts colony formation, a GT 1a wild-type replicon was treated with 20 nM Compound E, 10 nM Compound F, or a combination of 20 nM Compound E and 10 nM Compound F for 7 days, and then cultured with or without G418 in the absence of Compound F and Compound E (FIG. 3). Replicon cells treated with DMSO only was used as a control (FIG. 3, no compound). As shown in FIG. 3, the combination of Compound F plus Compound E was much more effective at eliminating HCV replicon, either in the presence or absence of G418, than either compound alone. These results clearly demonstrate that a combination of two NS5A compounds can synergistically inhibit HCV colony formation, yielding a higher genetic barrier for development of resistance.

While the invention has been described in connection with specific embodiments therefore, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims. All references cited herein are expressly incorporated in their entirety.

1. A method for identifying NS5A-targeting compounds that in combinations demonstrate a synergistic inhibitory interaction toward wild-type and/or variants that reduce the potency of the NS5A-targeting inhibitors alone comprising the steps of:
(a) screening a compound library for NS5A targeting compounds and
(b) comparing the amount of HCV inhibition of a NS5A-targeting inhibitor in the presence and absence of a fixed concentration of a NS5A-targeting compound and
wherein the second NS5A-targeting compound may or may not demonstrate HCV NS5A inhibitory activity when assayed alone and wherein the combination of the NS5A-targeting compounds demonstrate a synergistic inhibition of HCV replicon or virus replication.

2. The method of claim 1 wherein the compound library is screened in cells containing a HCV sequence derived from any genotype.

3. The method of claim 1 wherein the HCV inhibitory activity is determined by using a cell-based method or a biochemical surrogate.

4. The method of claim 2 wherein the HCV sequence is selected from the group consisting of wild-type or sequences carrying NS5A resistant variants.

5. The method of claim 2 wherein said HCV sequence is genotype 1b carrying a Y93H single amino acid substitution in NS5A.

6. The method of claim 2 wherein said HCV sequence is genotype 1b carrying a L31V single amino acid substitution in NS5A.

7. The method of claim 2 wherein said HCV sequence is genotype 1a carrying a M28T single amino acid substitution in NS5A.

8. The method of claim 2 wherein said HCV sequence is genotype 1a carrying a Q30R single amino acid substitution in NS5A.

9. The method of claim 2 wherein said HCV sequence is genotype 1a carrying a L31V single amino acid substitution in NS5A.

10. The method of claim 2 wherein said HCV sequence is genotype 1a carrying a Y93H single amino acid substitution in NS5A.

11. The method of claim 2 wherein said HCV sequence is genotype 1a carrying a Q30H single amino acid substitution in NS5A.

12. The method of claim 2 wherein said HCV sequence is genotype 1a carrying a Q30E single amino acid substitution in NS5A.

13. The method of claim 2 wherein said HCV sequence is genotype 1a carrying a Y93N single amino acid substitution in NS5A.

14. The method of claim 2 wherein said HCV sequence is genotype 1a carrying amino acid substitutions at both L31V and Y93H in NS5A.

15. The method of claim 2 wherein said HCV sequence is genotype 1a carrying amino acid substitutions at both M28T and Q30H in NS5A.

16. The method of claim 2 wherein said HCV sequence is genotype 1a carrying amino acid substitutions at both Q30R and H58D in NS5A.

17. The method of claim 2 wherein said HCV sequence is genotype 1a carrying amino acid substitutions at both Q30H and Y93H in NS5A.

18. The method of claim 2 wherein said HCV sequence is genotype 1a carrying amino acid substitutions at both Q30R and E62D in NS5A.

19. The method of claim 2 wherein said HCV sequence is genotype 1b carrying amino acid substitutions at both L31V and Y93H in NS5A.

20. The method of claim 3, wherein said cell-based method is a transient replication assay.

21. The method of claim 3, wherein said cell-based method is a FRET assay.

22. The method of claim 3, wherein said cell-based method is a luciferase assay.

23. The method of claim 3, wherein said cell-based method is a colony formation assay.

24. The method of claim 3, wherein said cell-based method is a Western blot assay.

25. The method of claim 3, wherein said cell-based method is a Taqman assay.

26. The method of claim 3, wherein said cell-based method is an ELISA assay.

27. The method of claim 3, wherein said biochemical surrogate is a NS5A hyperphosphorylation assay.

28. The method of claim 1, wherein the HCV inhibitory activity is increased by at least 3 fold.
